(19) United States
(12) Patent Application Publication
(10) Pub. No.: US 2009/0311220 A1
(43) Pub. Date: Dec. 17, 2009

(54) Bone forming compound composed of a mixture of osteoblast and biomatrix and method for producing the same

(76) Inventors: Jae-Deog Jang, Seoul (KR); Hyun-Shin Park, Seoul (KR); Cheong-Ho Chang, Seoul (KR); Soo-Jin Jung, Gyeonggi-Do (KR); Sae-Bom Lee, Seoul (KR); Chang-Kwon Ko, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 11/921,217

(22) PCT Filed: Jun. 27, 2005
(86) PCT No.: PCT/KR2005/002006

§ 371 Date: Nov. 29, 2007

(30) Foreign Application Priority Data
Jun. 13, 2005 (KR) 10-2005-0050447

(51) Int. Cl. A61K 35/32; A61P 19/00

(52) U.S. Cl. 424/93.7

(57) ABSTRACT

A compound for bone forming and a method for producing the same are provided using a mixture of an osteoblast and a bio-matrix. The method comprises: isolating osteoblasts from bone tissue and culturing the isolated osteoblasts to prepare an osteoblast suspension; and mixing the resulting osteoblast suspension with a bio-matrix to prepare an osteoblast therapeutic agent. A bone formation method is provided that results in no clinical graft rejection, and is capable of achieving effective and rapid bone formation via injection of a compound which has been pre-shaped to a certain extent, so as to alleviate problems associated with bone tissue formation in unwanted regions resulting from escape of injected osteoblasts from the targeted site for bone formation and then propagation thereof to other sites via the blood stream, which are caused by injection of an osteoblast suspension.

### BACKGROUND OF THE INVENTION

1. Field of the Invention

The present invention relates to a compound for bone formation using a mixture of osteoblasts and bio-matrix, and a method for preparing the same. More specifically, the present invention relates to a compound for bone formation using a mixture of osteoblasts and a bio-matrix that can be transplanted for bone formation when treating bone defects or into a region in need of reinforcement of the bone, and a method for preparing the same.

2. Related Prior Art

According to reports issued by the World Health Organization (WHO), over one half of people over the age of 65 suffer from chronic bone diseases, and the incidence of bone fracture related to osteoporosis has doubled over the past decade. These figures correspond to approximately 40% of women 50 and older.

In the USA, roughly 5.6 million people suffer bone fractures each year and 3.1 million of them have surgical operations. According to a statistical report by Medical Data International (MDI) in 1995, about 426,000 bone graft surgeries were conducted. Costs for bone grafting are estimated to be approximately 800 million US dollars per year throughout the world. In 1995, the breakdown of bone grafting methods was 58% autografting, 34% allografting and 8% synthetic material transplantation.

Generally, a simple (closed) bone fracture may be sufficiently healed by casting the bone for several weeks, but a severe bone fracture or bone defect requires bone grafting.

However, autografting disadvantageously may cause severe pain in a bone-harvested region, may require a long period of time for recovery after the surgical operation for bone grafting, and has suffered from a great deal of difficulty to secure donated bone for bone transplantation.

Meanwhile, allografting also suffers from fatal disadvantages such as weakening of bone strength during a sterilization process, the occurrence of graft rejection, and the possibility of transmission of contagious diseases such as hepatitis and AIDS.

In another available method for bone grafting, metals coated with biologically active or biologically inactive ceramic materials are widely used as supporting materials in orthopedic surgery, but there is a great deal of difficulty to use metals as bone transplants due to problems such as corrosion of metals, wear of the ceramic-metal surface, and severe formation of fibrose tissues at surfaces of the bones and transplants.

For bone-formation factors, although a great deal of studies have been made on various factors after Urist and Mclean (1952) issued a publication of the effects of bone formation proteins on bone formation, the production process thereof is very complicated and expensive, and has low efficiency, thus resulting in a low yield and ultimately limiting the application thereof for practical clinical uses.

Meanwhile, bone marrow injection is a technique based on the assertion, proposed by Huggins (1931), Friedenstein (1973), and Ashton (1980), that osteoprogenitor cells from bone marrow induce and facilitate bone formation. Bone marrow injection is generally carried out alone for healing bone fractures, but is also carried out in combination with bone grafting. Unlike other bone grafting techniques, this bone marrow injection does not involve surgical skin incision for donor parts and thus is significantly advantageous due to the absence of problems associated with securing donor parts and it has no complications or adverse side effects.

Thereafter, even though some excellent results have been published related to a great number of clinical application cases, bone marrow injection is disadvantageous due to its unsound theoretical basis, as evidenced by considerable numbers of outcomes that are not consistent, because the amount of bone marrow collectable from one site is limited, and because there is a significantly limited number of osteoprogenitor cells contained in bone marrow.

As such, a novel bone formation and transplantation method by culturing and amplifying osteoprogenitor cells into sufficient numbers of osteoblast cells, mixing the cultured cells with a bio-matrix and injecting the resulting mixture into a bone-formation region is a highly effective method, bringing significant advantages and beneficial effects as compared to conventional autografting, allografting and bone marrow injection. Thus, the field of tissue engineering in this manner is receiving a great deal of attention in bone regeneration therapies.

### SUMMARY OF THE INVENTION

Therefore, the present invention has been made in view of the problems associated with implants and conventional bone grafting techniques as discussed above, and it is an object of the present invention to provide a compound for bone formation using a mixture of osteoblasts and bio-matrix, and a method for preparing the same, that results in no clinical graft rejection via injection of osteoblasts and bio-matrix mixture for bone formation into a site where bone formation is sought, and that is capable of achieving effective and rapid bone formation via injection of a compound which has been pre-shaped to a certain extent, so as to alleviate problems associated with the bone tissue formation in unwanted regions resulting from escape of injected osteoblasts from the desired site for bone formation and then propagation thereof to other sites via the blood stream, which may be caused by injection of an osteoblast suspension.

In accordance with an aspect of the present invention, the above and other objects can be accomplished by the provision of a method for preparing a compound for bone formation, comprising:

isolating osteoblasts and their precursor cells from a bone tissue and culturing/proliferating the isolated osteoblasts and their precursor cells in DMEM (Dulbecco's Modified Eagle's Medium) or α-MEM (Minimum Essential Medium, Alpha Modification) to prepare an osteoblast suspension; and

mixing the resulting osteoblast suspension with a bio-matrix to prepare an osteoblast therapeutic agent.

Herein, the preparation step of the therapeutic agent further includes mixing the osteoblast mixed solution, in which the bio-matrix was mixed in the osteoblast suspension, with a coagulant.

Herein, the mixing step includes mixing the osteoblast solution with 10 to 100 IU/mL of thrombin as a coagulant; and mixing the resulting solution containing thrombin with 20 to 100 mg/mL of fibrinogen as the coagulant.

Herein, the bio-matrix is collagen, hydroxyapatite or a mixture thereof.

Herein, collagen may be added in an amount of 67 μg/mL to 20 mg/mL to the osteoblast suspension, and hydroxyapatite may be added in an amount of 30 μg/mL to 3.4 mg/mL to the osteoblast suspension.

Collagen is neutralized to a neutral pH by addition of a neutralization solution prior to mixing with the osteoblast suspension.

Prior to adding it to the solution of the osteoblast and bio-matrix as the thrombin component, lyophilized thrombin is dissolved in liquid DMEM or α-MEM to which phosphate (PO43−) ions have been added as a bone mineral component, in an amount that is double that of the phosphate (PO43−) ions. Then, prior to adding it to the resulting osteoblast mixed solution as the fibrinogen component, lyophilized fibrinogen is dissolved in liquid DMEM or α-MEM to which calcium (Ca2+) ions have been added as a bone mineral component, in an amount that is double that of the calcium (Ca2+) ions.

In accordance with another aspect of the present invention, there is provided a compound for bone formation prepared by the above method for preparing a compound for bone formation using a mixture of an osteoblast and a bio-matrix.

In accordance with a compound for bone formation using a mixture of an osteoblast and a bio-matrix, and a method for preparing the same, having a construction as described above, it is possible to achieve bone formation that results in no clinical graft rejection via injection of an osteoblast and bio-matrix mixture for bone formation into a site where bone formation is sought, and to achieve effective and rapid bone formation via injection of a compound which has been pre-shaped to a certain extent, so as to alleviate problems associated with bone tissue formation in unwanted regions resulting from escape of injected osteoblasts from the desired site for bone formation and then propagation thereof to other sites via the blood stream, which are caused by injection of an osteoblast suspension.

### BRIEF DESCRIPTION OF THE DRAWINGS

FIG. 1 is a bar graph showing a compound ratio of bone.

FIG. 2 is a process flow chart illustrating a method for preparing a compound for bone formation using a mixture of an osteoblast and a bio-matrix in accordance with the present invention.

FIG. 3 is a photograph illustrating subcutaneous injection of an osteoblast therapeutic agent, which is a mixture of an osteoblast and a bio-matrix, into an immunodeficient mouse.

FIG. 4 is a photograph of an immunodeficient mouse taken 4 weeks after transplantation of an osteoblast therapeutic agent, which is a mixture of an osteoblast and a bio-matrix.

FIG. 5 is a photograph of a transplant taken 4 weeks after transplantation of an osteoblast therapeutic agent, which is a mixture of an osteoblast and a bio-matrix, into an immunodeficient mouse.

FIG. 6 is an autoradiograph of an immunodeficient mouse taken 4 weeks after transplantation of an osteoblast therapeutic agent, which is a mixture of an osteoblast and a bio-matrix.

FIG. 7 is a photograph of a tissue section stained with Hematoxylin-Eosin capable of confirming bone formation, taken 8 weeks after transplantation of an osteoblast therapeutic agent, which is a mixture of an osteoblast and a bio-matrix, into an immunodeficient mouse.

FIG. 8 is a photograph of a tissue section stained with Masson's Trichrome, taken 8 weeks after transplantation of an osteoblast therapeutic agent, which is a mixture of an osteoblast and a bio-matrix, into an immunodeficient mouse.

### DETAILED DESCRIPTION OF THE PREFERRED EMBODIMENTS

Hereinafter, a method for preparing a compound for bone formation using a mixture of an osteoblast and a bio-matrix in accordance with the present invention will be described in more detail with reference to the accompanying drawings.

In order to overcome disadvantages exhibited by conventional therapies for treating bone-related disorders and diseases, such as implants and bone grafting, a technique of transplanting autologous osteoblasts that has no adverse side effects on an affected part, via culturing of osteoblasts, has been developed as an ultimate treatment method in conjunction with improvement in conventional therapies.

However, for achieving faster bone formation, it is necessary to mix osteoblasts with a matrix through which more diverse lesions can be treated, and thereby it is possible to treat more bone diseases that do not achieve therapeutic benefits by injection of a liquid osteoblast suspension consisting of osteoblasts only.

It can be said that the osteoblast therapeutic agent, which is a mixture of an osteoblast and a bio-matrix, is a more advanced version of an injection method of a liquid osteoblast suspension which is based on cell therapy. The conventional injection method of a liquid osteoblast suspension, which involves transplanting osteoblasts alone, can only treat limited types of bone defects and also suffers from problems associated with the occurence of bone tissue formation in unwanted regions resulting from escape of injected osteoblasts from desired bone-forming sites and then propagation thereof to other sites via the blood stream. In contrast, the osteoblast therapeutic agent in accordance with the present invention is made up of the osteoblast and bio-matrix mixture and therefore can also more rapidly and effectively treat a broader range of bone defects and severe bone diseases.

Bones are composed of bone cells and large quantities of bone matrices present between cells. Most parts of bone matrices are comprised of organic components (35%) made up of collagen fibers and inorganic components (65%) made up of calcium and phosphate.

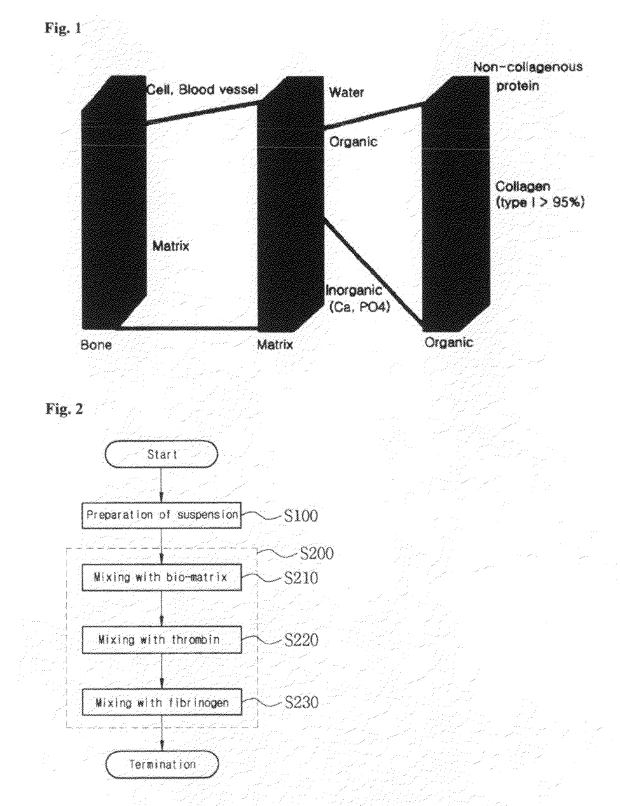

Bones, as shown in FIG. 1, are biosynthetic substances and are composed of minerals, collagen, moisture, non-collagenous proteins, lipids, vascular components and cells, in order of the highest compound ratio to the lowest.

The inorganic matter making up the largest portion of bone composition is an analogue of hydroxyapatite (HA) which is a geological mineral.

Bone apatite is generally deficient in calcium and hydroxyl groups, which are replaced by numerous impurities including carbonate, magnesium, potassium, boron, phosphate and citrate, the most abundant of which is carbonate.

The content of carbonate in the bone minerals increases with maturation of the bones. Carbonate replaces hydroxyl or phosphate groups, or may be adsorbed on the surface of bone apatite.

The second most abundant constituent of bones is collagen, mainly type I collagen. Collagen imparts elasticity and flexibility to bones and offers direction for matrix constitution.

According to the embodiment of the present invention, osteoblasts are mixed with the bio-matrix and the resulting mixture should be injected into regions suffering from bone diseases, and collagen and hydroxyapatite, which are bone components, are mixed and injected into lesions. Therefore, it is possible to secure physical properties of bones in advance and thereby realize faster bone formation and adequately mineralized bones.

In mixing the osteoblasts, a main component of the bone matrix, with the bio-matrix, one of the most crucial factors is stability. In order to ensure sufficient numbers of the cells within the matrix, the survival rate of cells should be enhanced. In addition, it is also important to increase the engrafting rate of the cells after performing transplantation thereof.

FIG. 2 is a process flow chart illustrating a method for preparing a compound for bone formation using a mixture of an osteoblast and a bio-matrix in accordance with the present invention.

Referring to FIG. 2, the method for preparing a compound for bone formation in accordance with the present invention comprises the steps of isolating osteoblasts from a bone tissue and culturing/proliferating the isolated osteoblasts in DMEM (Dulbecco's Modified Eagle's Medium) or α-MEM (Minimum Essential Medium, Alpha Modification) to prepare an osteoblast suspension (S100); and mixing the resulting osteoblast suspension with a bio-matrix to prepare an osteoblast therapeutic agent (S200).

Herein, the number of osteoblasts in the osteoblast suspension may vary significantly depending upon sizes and locations of lesions of interest. Typically, the number of osteoblasts is preferably in a range of 1×10̂6 to 1.2×10̂7 cells, although a higher number of cells may be used.

Herein, the preparation step (S200) of the therapeutic agent further includes mixing the osteoblast/bio-matrix solution, in which the bio-matrix was mixed in the osteoblast suspension, with a coagulant.

Herein, the mixing step includes mixing the osteoblast/bio-matrix solution with 10 to 100 IU/mL of thrombin as the coagulant (S220), and mixing the resulting solution containing thrombin with 20 to 100 mg/mL of fibrinogen as the coagulant (S230).

As the bio-matrix, collagen, hydroxyapatite or a mixture thereof may be used. When a mixture of collagen and hydroxyapatite is used as the bio-matrix, 67 μg/mL to 20 mg/mL of collagen is mixed with 30 μg/mL to 3.4 mg/mL of hydroxyapatite.

Meanwhile, a minimal concentration of collagen was optimal to induce cell differentiation and proliferation when collagen was used as a coating material for coating a culture vessel in culturing osteoblasts. When the collagen concentration applied at this time was applied as the matrix component, it could be confirmed that when injecting the compound in which osteoblasts and bio-matrix were mixed with each other, the collagen components, which were injected before the osteoblasts expressed collagen, form a basic matrix network, resulting in cell differentiation and rapid mineralization which consequently leads to rapid bone formation with a minimal amount of collagen. Meanwhile, where collagen was used as a sole matrix, a collagen concentration equal to or higher than 3 mg/mL (0.3%) leads to gelation under predetermined conditions, thereby forming a matrix. In contrast, a maximal concentration of 20 mg/mL of collagen results in a sharp decrease of fluidity thereof and thus osteoblasts are not homogeneously dispersed in the collagen matrix and it is thus difficult to apply the collagen as a cell matrix.

Further, hydroxyapatite should be added in a concentration such that hydroxyapatite promotes mineralization in a matrix mixture. Where an excess amount of hydroxyapatite is contained, this may inhibit cell differentiation due to spatial restriction which occurs due to occupation of the cell matrix mixture by hydroxyapatite. Therefore, in the present invention, hydroxyapatite was applied in a minimal concentration at which hydroxyapatite can serve as a nucleus for mineralization in the matrix component, and in an optimal concentration to promote mineralization.

On the other hand, collagen is neutralized to a neutral pH by addition of a neutralization solution prior to mixing with the osteoblast suspension.

In addition, prior to adding it to the mixed solution of the osteoblasts and bio-matrix as the thrombin comonent, lyophilized thrombin is dissolved in liquid DMEM or α-MEM to which phosphate (PO43−) ions have been added, in an amount that is double that of the phosphate (PO43−) ions. Then, prior to adding it to the resulting osteoblast mixed solution as the fibrinogen component, lyophilized fibrinogen is dissolved in liquid DMEM or α-MEM to which calcium (Ca2+) ions have been added, in an amount that is double that of the calcium (Ca2+) ions. Herein, phosphate ions and calcium ions exhibit no cytotoxic effects on osteoblasts and act to supplement the bone mineral components.

Thrombin as the coagulant is mixed in an amount of 10 to 100 IU/mL in the mixed solution, and fibrinogen as the coagulant is mixed in an amount of 20 to 100 mg/mL in the resulting mixture containing thrombin.

Meanwhile, the concentration of thrombin determines the polymerization time of fibrin. Therefore, the polymerization time of fibrin can be reduced within a range of 4 hours to 3 sec, depending upon the concentration of thrombin. The concentration of thrombin is such that the compound of the present invention is shaped to prevent a mixed compound of osteoblasts and bio-matrix from leaking away from sites of interest when injected into a site for bone formation, injection of the compound into the affected site leads to rapid polymerization, and an optimal fibrin pore matrix for bone formation by osteoblasts is formed.

### EXAMPLES

Hereinafter, effects of a compound for bone formation using a mixture of an osteoblast and a bio-matrix in accordance with an example of the present invention will be described in more detail with reference to the accompanying drawings.

### Example 1

Firstly, osteoblasts and their precursor cells were isolated from the corresponding tissues, and cultured and proliferated in DMEM or α-MEM for 4 weeks, thereby preparing an osteoblast suspension composed of DMEM or α-MEM. As a bio-matrix to be mixed therein, collagen was prepared.

Then, the resulting osteoblast suspension was mixed with collagen to thereby prepare a total of 1 mL of a compound for bone formation.

### Example 2

Osteoblasts and their precursor cells were isolated from the corresponding tissues, and cultured and proliferated in DMEM or α-MEM for 4 weeks, thereby preparing an osteoblast suspension composed of DMEM or α-MEM. As bio-matrices, collagen and hydroxyapatite were prepared. A 1:10 volume ratio of hydroxyapatite was added to the osteoblast suspension and mixed well. As the bio-matrix to be mixed therein, a 2:5 volume ratio of collagen was prepared.

The osteoblast suspension was mixed with collagen to prepare a total of 1 mL of a compound for bone formation.

### Example 3

Osteoblasts and their precursor cells were isolated from the corresponding tissues, and cultured and proliferated in DMEM or α-MEM for 4 weeks, thereby preparing an osteoblast suspension composed of DMEM or α-MEM. As bio-matrices, collagen and hydroxyapatite were prepared. A 2:5 volume ratio of collagen and a 1:10 volume ratio of hydroxyapatite were added to the osteoblast suspension and mixed well, thereby forming an osteoblast therapeutic agent mixed with bio-matrices.

After preparing a medical-grade fibrin glue set at room temperature, fibrinogen was dissolved by adding a proper quantity of liquid DMEM or α-MEM to a vial containing lyophilized fibrinogen. In addition, thrombin was also dissolved by adding a proper quantity of liquid DMEM or α-MEM to a vial containing the lyophilized thrombin.

Then, a 1:10 volume ratio of the dissolved thrombin was added to the liquid osteoblast suspension in which bio-matrices were mixed, and the resulting mixture was mixed well.

The osteoblast suspension mixed with thrombin and bio-matrices was mixed with an equal amount of dissolved fibrinogen to prepare a total of 1 mL of a compound for bone formation.

### Example 4

Osteoblasts and their precursor cells were isolated from the corresponding tissues, and cultured and proliferated in DMEM or α-MEM for 4 weeks, thereby preparing an osteoblast suspension composed of DMEM or α-MEM. As bio-matrices, collagen and hydroxyapatite were prepared. A 2:5 volume ratio of collagen and a 1:10 volume ratio of hydroxyapatite were added to the osteoblast suspension and mixed well, thereby forming an osteoblast therapeutic agent mixed with the bio-matrices.

After preparing a medical-grade fibrin glue set at room temperature, fibrinogen was dissolved by adding a proper quantity of liquid DMEM or α-MEM to a vial containing lyophilized fibrinogen. In addition, thrombin was also dissolved by adding a proper quantity of liquid DMEM or α-MEM to a vial containing the lyophilized thrombin.

At this time, the liquid DMEM or α-MEM was used to which phosphate (PO43−) ions had been added to a concentration of 2 mg/mL. In addition, a proper quantity of liquid DMEM or α-MEM was added to a vial containing the lyophilized thrombin, thereby dissolving the thrombin. At this time, the liquid DMEM or α-MEM was used to which calcium (Ca2+) ions had been added to a concentration of 4 mg/mL. Amounts of phosphate ions (PO43−) and calcium ions (Ca2+) used herein are optimal amounts which do not exhibit cytotoxic effects on osteoblasts and serve as additives for the culture solution to thereby supplement bone mineral components.

Then, the dissolved thrombin was added in a 1:10 volume ratio to the liquid osteoblast suspension mixed with bio-matrices, and mixed well.

The osteoblast suspension containing thrombin and bio-matrices mixed therein was mixed with an equal amount of dissolved fibrinogen to prepare a total of 1 mL of a compound for bone formation.

### Results:

Fifteen (15) immunodeficient mice were divided into 6 groups (triple trial). Then, an osteoblast therapeutic agent, which is a mixture of an osteoblast and a bio-matrix, was prepared and injected into the scapula of nude mice via subcutaneous injection. One (1) mL of the osteoblast therapeutic agent was injected into the hypoderm of each immunodeficient mouse. The results were confirmed 4 weeks and 8 weeks after injection.

TABLE 1

| | Group 1 | Group 2 | Group 3 |
|---|---|---|---|
| Cell number (cells) | 1 × 10\hat{ }6 cells | 6 × 10\hat{ }6 cells | 1.2 × 10\hat{ }7 cells |
| Injection volume | 1 mL | 1 mL | 1 mL |
| (cc) | | | |

Table 1 shows respective osteoblast therapeutic agents, which are mixtures of osteoblasts and bio-matrices, having the same bio-matrix compound but different numbers of osteoblasts that were transplanted into immunodeficient mice.

TABLE 2

| | Group 1 | Group 2 | Group 3 |
|---|---|---|---|
| Size volume (cc) | 0.26 | 0.24 | 0.25 |
| Bone formation | Good | Good | Good |

Here, a bone formation grade is rated in order of excellent/good/fair/poor.

Eight weeks after transplanting the osteoblast therapeutic agents as given in Table 1, the results for bone formation thus obtained are shown in Table 2.

TABLE 3

| | Group 4 | Group 5 | Group 6 |
|---|---|---|---|
| Cell number (cells) | 6 × 10\hat{ }6 cells | 6 × 10\hat{ }6 cells | 6 × 10\hat{ }6 cells |
| Collagen vol. | 1:5 | 2:5 | 1 |
| Injection volume | 1 mL | 1 mL | 1 mL |
| (cc) | | | |

Table 3 shows the respective osteoblast therapeutic agents having different amounts of bio-matrices added, which are mixtures of osteoblasts and bio-matrices, that were transplanted into immunodeficient mice.

TABLE 4

| | Group 4 | Group 5 | Group 6 |
|---|---|---|---|
| Size volume (cc) | 0.24 | 0.52 | 0.50 |
| Bone formation | Good | Excellent | Excellent |

Here, a bone formation grade is rated in order of excellent/good/fair/poor.

Eight weeks after transplanting the osteoblast therapeutic agents as given in Table 3, the results for bone formation thus obtained are shown in Table 4.

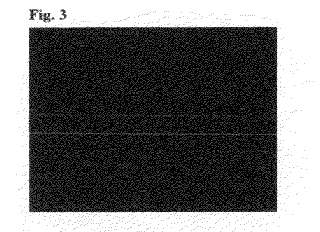

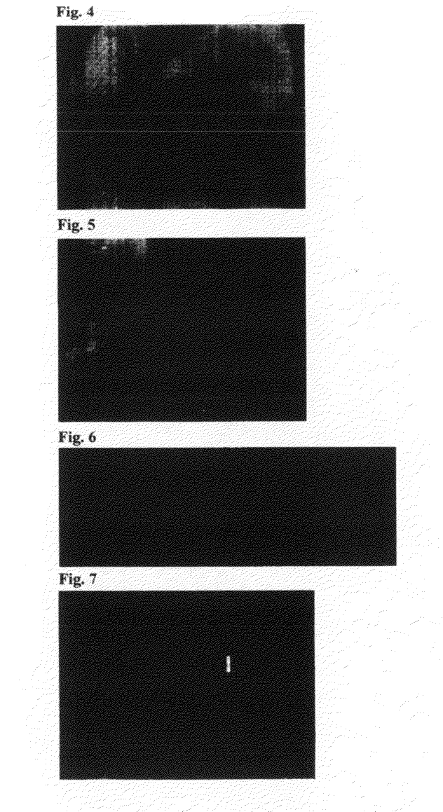

FIG. 3 is a photograph illustrating subcutaneous injection of an osteoblast therapeutic agent, which is a mixture of an osteoblast and a bio-matrix, into an immunodeficient mouse. FIG. 4 is a photograph of bone formation (observed as a bulged portion circumscribed by a dotted line) in an immunodeficient mouse taken 4 weeks after transplantation of an osteoblast therapeutic agent, which is a mixture of an osteoblast and a bio-matrix.

FIG. 5 is a photograph of a bone-formation transplant taken 4 weeks after transplantation of an osteoblast therapeutic agent, which is a mixture of an osteoblast and a bio-matrix, into an immunodeficient mouse, thus confirming that formation of blood vessels was induced around the transplant. FIG. 6 is an autoradiograph of an immunodeficient mouse taken 4 weeks after transplantation of an osteoblast therapeutic agent, which is a mixture of an osteoblast and a bio-matrix, wherein it was observed that a new bone was formed on the backbone, as circumscribed by a dotted-line circle.

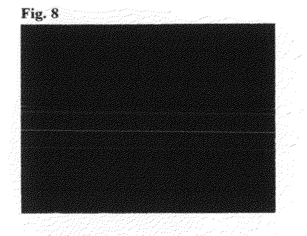

FIG. 7 is a photograph of a tissue section stained with Hematoxylin-Eosin capable of confirming bone formation, taken 8 weeks after transplantation of an osteoblast therapeutic agent, which is a mixture of an osteoblast and a bio-matrix, into an immunodeficient mouse. As can be seen, osteoid (represented by a block arrow) is observed and osteoblasts distributed within Lacuna (represented by a white arrow) can also be confirmed.

FIG. 8 is a photograph of a tissue section stained with Masson's Trichrome, taken 8 weeks after transplantation of an osteoblast therapeutic agent, which is a mixture of an osteoblast and a bio-matrix, into an immunodeficient mouse, and it can be confirmed that bone formation occurred in conjunction with collagen along predetermined patterns, thus showing penetration of blood vessels between matrices.

Thus, in accordance with the present invention, there is provided a bone formation method that results in no clinical graft rejection via injection of an osteoblast and bio-matrix mixture into a site where bone formation is sought, and that is capable of achieving effective and rapid bone formation via injection of a compound which has been pre-shaped to a certain extent.

In accordance with the present. invention, a compound for bone formation using a mixture of an osteoblast and a bio-matrix, and a method for preparing the same, having a construction as described above, enable realization of bone formation resulting in no clinical graft rejection via injection of an osteoblast and bio-matrix mixture for bone formation into a site where bone formation is sought, and are capable of achieving effective and rapid bone formation via injection of a compound which has been pre-shaped to a certain extent, so as to alleviate problems associated with bone tissue formation in unwanted regions resulting from escape of injected osteoblasts from the targeted site for bone formation and then propagation thereof to other sites via the blood stream, which are caused by injection of an osteoblast suspension.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1-8. (canceled)

9. A method for preparing a compound for bone formation using a mixture of an osteoblast and a bio-matrix, comprising:
isolating osteoblasts and their precursor cells from a bone tissue and culturing/proliferating the isolated osteoblasts and their precursor cells in DMEM (Dulbecco's Modified Eagle's Medium) or α-MEM (Minimum Essential Medium, Alpha Modification) to prepare an osteoblast suspension; and
mixing the resulting osteoblast suspension with a bio-matrix to prepare an osteoblast therapeutic agent.

10. The method according to claim 9, wherein the preparation step of the therapeutic agent further includes mixing the osteoblast solution, in which the bio-matrix was mixed into the osteoblast suspension, with a coagulant.

11. The method according to claim 10, wherein the mixing step includes:
mixing the osteoblast mixed solution with 10 to 100 IU/mL of thrombin as the coagulant; and
mixing the resulting solution containing thrombin with 20 to 100 mg/mL of fibrinogen as the coagulant.

12. The method according to claim 9, wherein the bio-matrix is collagen, hydroxyapatite or a mixture thereof.

13. The method according to claim 12, wherein collagen is added in an amount of 67 μg/mL to 20 mg/mL to the osteoblast suspension, and hydroxyapatite is added in an amount of 30 μg/mL to 3.4 mg/mL to the osteoblast suspension.

14. The method according to claim 13, wherein collagen is neutralized to a neutral pH by addition of a neutralization solution prior to mixing with the osteoblast suspension.

15. The method according to claim 11, wherein prior to adding it to the mixture of the osteoblast and bio-matrix as a thrombin component, lyophilized thrombin is dissolved in liquid DMEM or α-MEM to which phosphate (PO43−) ions have been added as a bone mineral component, in an amount that is double that of the phosphate (PO43−) ions; and
prior to adding it to the resulting osteoblast mixed solution as a fibrinogen component, lyophilized fibrinogen is dissolved in liquid DMEM or α-MEM to which calcium (Ca2+) ions have been added as a bone mineral component, in an amount that is double that of the calcium (Ca2+) ions.

16. A preparation of compound for bone formation by using a mixture of an osteoblast and a bio-matrix comprising:
a means for isolating osteoblasts and their precursor cells from a bone tissue and culturing/proliferating the isolated osteoblasts and their precursor cells in DMEM (Dulbecco's Modified Eagle's Medium) or α-MEM (Minimum Essential Medium, Alpha Modification) to prepare an osteoblast suspension; and
a means for mixing the resulting osteoblast suspension with a bio-matrix to prepare an osteoblast therapeutic agent.
